(19) United States
(12) Patent Application Publication
(10) Pub. No.: US 2026/0225976 A1
(43) Pub. Date: Aug. 6, 2026

(54) ETHYLENE OLIGOMERIZATION METHOD WITH REDUCED POLYMER GENERATION AND IMPROVED PRODUCTIVITY

(71) Applicant: HANWHA TOTALENERGIES PETROCHEMICAL CO., LTD., Seosan-Si, Chungcheongnam-Do (KR)

(72) Inventors: Kyesung CHO, Seosan-si Chungcheongnam-do (KR); Sangjoon OH, Seosan-si Chungcheongnam-do (KR); Ho Sik CHANG, Yuseong-gu Daejeon (KR); Jinsuk LEE, Dongjak-gu Seoul (KR)

(21) Appl. No.: 19/147,249

(22) PCT Filed: Jan. 15, 2024
(86) PCT No.: PCT/KR2024/000693

§ 371 Date: Jul. 10, 2025

(30) Foreign Application Priority Data
May 19, 2023 (KR) 10-2023-0065046

(51) Int. Cl. C07C 11/107; B01J 31/12; B01J 31/14; C07C 2/30

(52) U.S. Cl. CPC C07C 11/107; B01J 31/122; B01J 31/143; C07C 2/30; B01J 2231/20; B01J 2531/62

(57) ABSTRACT

The present invention relates to an ethylene oligomerization method, and more specifically, to a method for continuous production of ethylene oligomerization, in which ethylene is oligomerized by reacting ethylene with a chromium complex, an organoaluminum compound, and an organozinc compound, wherein the method, by optimizing the conditions of reaction temperature and reaction pressure and the amount of the organozinc compound injection, can improve productivity and significantly reduce the amount of polymer generation.

### TECHNICAL FIELD

The present invention relates to an ethylene oligomerization method having reduced polymer production and improved productivity.

### BACKGROUND ART

Among ethylene oligomers, 1-hexene and 1-octene are materials which are used in large amounts as a comonomer when polymerizing polyolefins such as polyethylene, and as production of polyolefins using a homogeneous metallocene-based catalyst increases, a demand therefor is increasing steadily.

Conventionally, various 1-alkenes having about 4 to 30 carbons are produced by oligomerizing ethylene in a shell higher olefin process (SHOP) based on a nickel catalyst, and 1-hexene and/or 1-octene may be obtained by separately separating them therefrom.

Thereafter, a catalyst system which may produce 1-hexene or 1-octene in a high yield by increasing selectivity in an ethylene oligomerization reaction was developed.

As a representative example, a method for preparing a catalyst which may selectively produce 1-octene and 1-hexene using a chromium trivalent compound (CrCl₃ for Cr(acac)₃), iPrN(PPh₂)₂ which is a bisphosphine ligand, and methylaluminoxane (MAO) was reported in U.S. Pat. No. 7,511,183 B2.

However, it was reported that the catalyst system should use high-priced MAO in a large amount (Al/Cr=300-500) in order to implement a commercially viable level of activity and also has a problem of producing a large amount of polyethylene (PE) which seriously impairs process stability (Organometallics, 27, 5712-5716).

In addition, an oligomerization catalyst system has reduced catalytic activity at a high temperature to reduce productivity and selectivity of olefins, in particular 1-octene, and as production of by-products is increased, tube blockage and fouling occur leading to shut down inevitably, thereby causing serious problems in an olefin polymerization process.

Specifically, polyethylene produced as a by-product forms a polymer layer and another polymer layer is formed again on the formed polymer layer, which lowers a fluid velocity, and a polymer coating layer formed along a reactor wall acts as an insulator which adversely affects heat transfer. That is, tube blockage and fouling occur, and a second treatment is needed in order to remove a polymer layer, which causes frequent process shut down.

Therefore, an ethylene oligomerization method of an improved process which may prepare 1-hexene and 1-octene with high selectivity without reducing catalytic activity and also may significantly decrease a polyethylene production amount which impairs process stability is required.

### DISCLOSURE

### Technical Problem

An object of the present invention is to provide an ethylene oligomerization method which may produce 1-hexene and 1-octene with a high conversion rate and also may significantly reduce a polyethylene production amount which impairs process stability.

### Technical Solution

The present invention provides a continuous ethylene oligomerization production method including oligomerizing ethylene by reacting a chromium complex, an organoaluminum compound, and an organozinc compound with ethylene, which may improve productivity and significantly decrease a polymer production amount through optimization of the conditions of reaction temperature and reaction pressure, and the injection amount of an organozinc compound.

In one general aspect, an ethylene oligomerization method includes reacting a chromium complex represented by the following Chemical Formula 1, an organoaluminum compound represented by the following Chemical Formula 2, and an organozinc compound represented by the following Chemical Formula 3 with ethylene in the presence of an organic solvent to prepare an ethylene oligomer, wherein a mole ratio (Cr:Zn) between the chromium complex and the organozinc compound is 1:100 to 1500, and the reaction is performed at a temperature of 0 to 80° C. under a pressure of 1 to 100 bar:

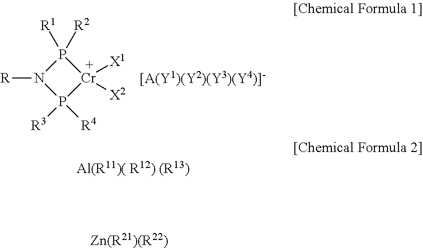

wherein
R is C1-C60 alkyl, C6-C60 aryl, or C2-C60 heteroaryl;
R¹ to R⁴ are independently of one another C1-C60 alkyl, C6-C60 aryl, or C2-C60 heteroaryl;
X¹ and X² are independently of each other a halogen, C1-C30 alkyl, C1-C30 alkylcarboxylate, acetylacetonate, or C1-C30 hydrocarbyl including one or more selected from ether and amino;
A is boron or aluminum;
Y¹ to Y⁴ are independently of one another fluorine-substituted C6-C60 aryl, fluorine-substituted C2-C60 heteroaryl, fluorine-substituted C6-C60 aryloxy, or fluorine-substituted C6-C60 alkoxy;
R¹¹ to R¹³ are independently of one another C4-C8 alkyl;
R²¹ to R²² are independently of each other C1-C8 alkyl; the alkyl, aryl, or heteroaryl of R and R¹ to R⁴ may be further substituted by one or more selected from C1-C30 alkyl, C6-C30 aryl, C1-C30 alkoxy, monoC1-C30 alkylamino, diC1-C30 alkylamino, monoC6-C30 arylamino, diC6-C30 arylamino, monoC1-C30 alkylsilyl, diC1-C30 alkylsilyl, triC1-C30 alkylsilyl, monoC6-C30 arylsilyl, diC6-C30 arylsilyl, and triC6-C30 arylsilyl; and the heteroaryl includes at least one heteroatom selected from the group consisting of O, N, S, Si, and P.

### Advantageous Effects

The ethylene oligomerization method according to an exemplary embodiment of the present invention may produce 1-hexene and 1-octene with high activity and selectivity, and also, may significantly decrease a polyethylene production amount without reducing catalytic activity.

### DESCRIPTION OF DRAWINGS

FIG. 1 is a photograph of the inside of a reactor after the reaction of Example 1 of the present invention.

FIG. 2 is a photograph of the inside of a reactor after the reaction of Comparative Example 1 of the present invention.

### BEST MODE

Hereinafter, some exemplary embodiments of the present disclosure will be described in detail with reference to the illustrative drawings.

In describing the present exemplary embodiments, when it is considered that detailed description of related well-known constructions or functions may obscure the gist of the present technical idea, the detailed description may be omitted.

When “comprise”, “have”, “consist of”, and the like mentioned in the present specification are used, other parts may be added unless “only” is used. When a constituent element is expressed in singular, it may include the case including plural unless particularly explicitly described.

In addition, in describing constituent elements of the present disclosure, terms such as first, second, A, B, (a), and (b) may be used. These terms are used only to differentiate the constituent elements from other constituent elements, and do not define the nature, sequence, order, number, or the like of the corresponding constituent elements.

In describing the positional relationship of the constituent elements, when it is described that two or more constituent elements are “linked”, “combined”, “connected”, or the like, the two or more constituent elements may be directly “linked”, “combined”, or “connected”, but it should be understood that the two or more constituents may be “linked”, “combined”, or “connected” by “intervening” another constituent element. Herein, another constituent element may be included in one or more of the “linked”, “combined”, or “connected” two or more constituent elements.

In describing time passing relationship related to constituent elements, operation methods, manufacturing methods, and the like, for example, when a temporal before-and-after relationship or a time passing before-and-after relationship is described by “after”, “following”, “subsequent to”, “before”, and the like, a non-continuous case may also be included unless “right” or “directly” is used.

Meanwhile, when the numerical number or corresponding information (e.g., level or the like) of a constituent element is mentioned, it may be construed that the numerical number or the corresponding information includes an error range which may be caused by various factors (e.g., process factors, internal or external impacts, noises, and the like), unless otherwise explicitly described.

The term “alkyl” used in the present specification (in the case of particularly not defining the number of carbon atoms) refers to a saturated straight chain or branched acyclic hydrocarbon having 1 to 60, preferably 1 to 30, in an exemplary embodiment, preferably 1 to 20, in an exemplary embodiment, preferably 1 to 10, in an exemplary embodiment, preferably 1 to 7 carbon atoms. “Lower alkyl” refers to straight chain or branched alkyl having 1 to 7, in an exemplary embodiment, preferably 1 to 5 carbon atoms.

Representative saturated straight chain alkyl includes methyl, ethyl, n-propyl, n-butyl, n-pentyl, n-hexyl, n-heptyl, n-octyl, n-nonyl, n-decyl, and the like, and saturated branched alkyl includes isopropyl, sec-butyl, isobutyl, tert-butyl, isopentyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2, 3-dimethylhexyl, 2, 4-dimethylhexyl, 2, 5-dimethylhexyl, 2, 2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2, 2-diethylhexyl, 3,3-diethylhexyl and the like.

In the present specification, when “C1-C10” is described, it means that the number of carbon atoms is 1 to 10. For example, C1-C10 alkyl refers to alkyl having 1 to 10 carbon atoms.

The term “aryl” used in the present specification refers to a carbocyclic aromatic group containing 5 to 10 ring atoms. A representative e example includes phenyl, tolyl, xylyl, naphthyl, tetrahydronaphthyl, anthracenyl, fluorenyl, indenyl, azulenyl, and the like, but is not limited thereto. The carbocyclic aromatic group may be selectively substituted.

The term “aryloxy” used in the present specification is RO—, in which R is the aryl defined above.

The term “fluorine-substituted aryl”, “fluorine-substituted aryloxy”, or “fluorine-substituted alkoxy” used in the present specification refer to an aryl, aryloxy, or alkoxy group in which one or more hydrogen atoms are substituted by a fluorine atom, respectively. For example, haloaryl includes-C6H4F, —C6H3F2, —C6HF4, and the like. Herein, aryl and halogen are as defined above, and alkoxy is as defined below.

The term “alkoxy” used in the present specification refers to—O-(alkyl) including —OCH₃, —OCH₂CH₃, —O(CH₂)₂CH₃, —O(CH₂)₃CH₃, —O(CH₂)₄CH₃, —O(CH₂)₅CH₃, and the like, in which alkyl is as defined above.

The term “lower alkoxy” used in the present specification refers to —O-(lower alkyl), in which the lower alkyl is as defined above.

The term “mono-alkylamino” used in the present specification refers to —NH(alkyl) including —NHCH₃, —NHCH₂CH₃, —NH(CH₂)₂CH₃, —NH(CH₂)₃CH₃, —NH(CH₂)₄CH₃, —NH(CH₂)₅CH₃, and the like, in which the alkyl is as defined above.

The term “di-alkylamino” used in the present specification refers to —N(alkyl) (alkyl) including —N(CH₃)₂, —N(CH₂CH₃)₂, —N((CH₂)₂CH₃)₂, —N(CH₃)(CH₂CH₃), and the like, in which alkyl is independently of each other the alkyl defined above.

The term “mono-alkylsilyl” used in the present specification refers to —SiH₂(alkyl) including —SiH₂CH₃, —SiH₂CH₃, —SiH₂(CH₂)₂CH₃, —SiH₂(CH₂)₃CH₃, —SiH₂(CH₂)₄CH₃, —SiH₂(CH₂)₅CH₃, and the like, in which the alkyl is as defined above.

The term “di-alkylsilyl” used in the present specification refers to —SiH(alkyl) (alkyl) including —SiH(CH₃)₂, —SiH(CH(CH₃)₂) (CH₃), —SiH((CH₂)₂CH₃)₂, —SiH(CH₃) (CH₂CH₃), and the like, in which alkyl is independently of each other the alkyl defined above.

The term “trialkylsilyl” used in the present specification refers to —Si(alkyl) (alkyl) (alkyl) including —Si(CH₃)₃, —Si(CH₂(CH₃)) 3, —Si((CH₂)₂CH₃)₃, —Si(CH₃)₂(CH₂CH₃), and the like, in which alkyl is independently of each other the alkyl defined above.

The terms “monoarylsilyl”, “diarylsilyl”, and “triarylsilyl” used in the present specification are substituents having aryl corresponding to alkyl in “monoalkylsilyl”, “dialkylsilyl”, or “trialkylsilyl”.

The term “heteroatom” used in the present specification refers to O, N, S, P, or Si unless otherwise described, but is not limited thereto.

Hereinafter, an ethylene oligomerization method according to an exemplary embodiment of the present invention will be described in detail.

The present invention provides a continuous ethylene oligomerization production method including performing ethylene oligomerization by reacting a chromium complex, an organoaluminum compound, and an organozinc compound with ethylene, which may improve productivity and significantly decrease a polymer production amount through optimization of the conditions of reaction temperature and reaction pressure, and the injection amount of an organozinc compound.

An exemplary embodiment provides an ethylene oligomerization method including reacting a chromium complex represented by the following Chemical Formula 1, an organoaluminum compound represented by the following Chemical Formula 2, and an organozinc compound represented by the following Chemical Formula 3 with ethylene in the presence of an organic solvent to prepare an ethylene oligomer, wherein a mole ratio (Cr:Zn) between the chromium complex and the organozinc compound is 1:100 to 1500, and the reaction is performed at a temperature of 0 to 80° C. under a pressure of 1 to 100 bar:

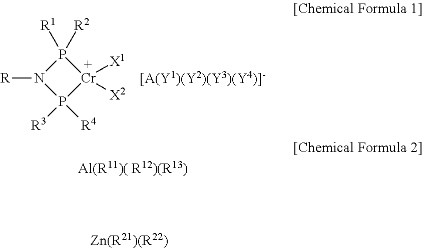

wherein
R is C1-C60 alkyl, C6-C60 aryl, or C2-C60 heteroaryl;
R¹ to R⁴ are independently of one another C1-C60 alkyl, C6-C60 aryl, or C2-C60 heteroaryl;
X¹ and X² are independently of each other a halogen, C1-C30 alkyl, C1-C30 alkylcarboxylate, acetylacetonate, or C1-C30 hydrocarbyl including one or more selected from ether and amino;
A is boron or aluminum;
Y¹ to Y⁴ are independently of one another fluorine-substituted C6-C60 aryl, fluorine-substituted C2-C60 heteroaryl, fluorine-substituted C6-C60 aryloxy, or fluorine-substituted C6-C60 alkoxy;
R¹¹ to R¹³ are independently of one another C4-C8 alkyl;
R²¹ to R²² are independently of each other C1-C8 alkyl;
the alkyl, aryl, or heteroaryl of R and R¹ to R⁴ may be further substituted by one or more selected from C1-C30 alkyl, C6-C30 aryl, C1-C30 alkoxy, monoC1-C30 alkylamino, diC1-C30 alkylamino, monoC6-C30 arylamino, diC6-C30 arylamino, monoC1-C30 alkylsilyl, diC1-C30 alkylsilyl, tric1-C30 alkylsilyl, monoC6-C30 arylsilyl, diC6-C30 arylsilyl, and tric6-C30 arylsilyl; and
the heteroaryl includes at least one heteroatom selected from the group consisting of O, N, S, Si, and P.

The ethylene oligomerization method of the present invention may dramatically suppress production of by-products such as polyolefin while maintaining catalytic activity simultaneously to maintain process stability, by using the chromium complex of Chemical Formula 1, the organoaluminum compound of Chemical formula 2, and the organozinc compound of Chemical Formula 3 and also simultaneously optimizing conditions of reaction temperature and reaction pressure and an injection amount of the organozinc compound. In addition, the ethylene oligomerization method of the present invention may have excellent catalytic activity, produce an ethylene oligomer with excellent selectivity and conversion rate even at a low temperature, and show high catalytic activity without using expensive conventional methylaluminoxane by using the specific chromium complex of Chemical Formula 1, and thus, ethylene oligomerization is allowed.

The ethylene oligomerization reaction according to an exemplary embodiment may be performed at a temperature of 20 to 80° C. under a pressure of 10 to 50 bar, specifically at a temperature of 40 to 60° C. under a pressure of 20 to 40 bar, and more specifically at a temperature of 40 to 50° under a pressure of 20 to 30 bar.

The ethylene oligomerization according to an exemplary embodiment may be performed in all reactors, but preferably, may be performed in a continuous stirred tank reactor (CSTR) or a plug flow reactor (PFR), more preferably in a continuous stirred tank reactor (CSTR).

When the reactor according to an exemplary embodiment is the continuous stirred tank reactor, it is more preferred that 90% or less, preferably 80% or less of the total volume of the reactor is maintained in a liquid phase.

In an exemplary embodiment, a mole ratio (Cr:Al) between the chromium complex of Chemical Formula 1 and the organoaluminum compound of Chemical Formula 2 may be 1:100 to 500, preferably 1:100 to 300, and more preferably 1:150 to 250.

In an exemplary embodiment, the mole ratio (Cr:Zn) between the chromium complex of Chemical Formula 1 and the organozinc compound of Chemical Formula 3 may be 1:100 to 1500, preferably 1:500 to 1300, and more preferably 1:700 to 1100.

The ethylene oligomerization method according to an exemplary embodiment is characterized by a specific combination of the specific chromium complex of Chemical Formula 1, the organoaluminum compound of Chemical Formula 2, and the organozinc compound of Chemical Formula 3, a specific mole ratio between the chromium complex and the organozinc compound, reaction temperature, and pressure.

In Chemical Formula 1 of the chromium complex according to an exemplary embodiment, R may be C1-C60 alkyl; R¹ to R⁴ may be independently of one another C6-C60 aryl; X¹ and X² may be independently of each other a halogen, C1-C30 alkyl, C1-C30 alkylcarboxylate, acetylacetonate, or C1-C30 alkoxy; A may be boron or aluminum; Y¹ to Y⁴ may be independently of one another fluorine-substituted C6-C60 aryl, fluorine-substituted C6-C60 aryloxy, or fluorine-substituted C6-C60 alkoxy; the alkyl of R and the aryl of R¹ to R⁴ may be further substituted by one or more selected from C1-C30 alkyl, C6-C30 aryl, C1-C30 alkoxy, monoC1-C30 alkylamino, diC1-C30 alkylamino, monoC6-C30 arylamino, diC6-C30 arylamino, monoalkylsilyl, diC1-C30 alkylsilyl, tric1-C30 alkylsilyl, monoC6-C30 arylsilyl, diC6-C30 arylsilyl, and tric6-C30 arylsilyl.

In Chemical Formula 1 of the chromium complex according to an exemplary embodiment, R may be C1-C30 alkyl; R¹ to R⁴ may be independently of one another C6-C30 aryl; X¹ and X² may be independently of each other a halogen, C1-C30 alkyl, C1-C30 alkylcarboxylate, acetylacetonate, or C1-C30 alkoxy; A may be boron; Y¹ to Y⁴ may be independently of one another fluorine-substituted C6-C30 aryl, fluorine-substituted C6-C30 aryloxy, or fluorine-substituted C6-C30 alkoxy; and the alkyl of R and the aryl of R¹ to R⁴ may be further substituted by one or more selected from C1-C20 alkyl, C6-C20 aryl, C1-C20 alkoxy, monoC1-C20 alkylamino, diC1-C20 alkylamino, monoC6-C20 arylamino, diC6-C20 arylamino, monoC1-C20 alkylsilyl, diC1-C20 alkylsilyl, tric1-C20 alkylsilyl, monoC6-C20 arylsilyl, diC6-C20 arylsilyl, and tric6-C20 arylsilyl,
more preferably, R may be C1-C20 alkyl; R¹ to R⁴ may be independently of one another C6-C20 aryl; X¹ and X² may be independently of each other a halogen, C1-C20 alkyl, C1-C20 alkylcarboxylate, or acetylacetonate; A may be boron; Y¹ to Y⁴ may be independently of one another fluorine-substituted C6-C20 aryl, fluorine-substituted C6-C20 aryloxy, or fluorine-substituted C6-C20 alkoxy; and the alkyl of R and the aryl of R¹ to R⁴ may be further substituted by one or more selected from C1-C10 alkyl, C6-C12 aryl, C1-C10 alkoxy, monoC1-C10 alkylamino, diC1-C10 alkylamino, monoC6-C12 arylamino, diC6-C12 arylamino, monoC1-C10 alkylsilyl, diC1-C10 alkylsilyl, and tric1-C10 alkylsilyl, and
more preferably, R may be C1-C10 alkyl; R¹ to R⁴ may be independently of one another C6-C12 aryl; X¹ and X² may be independently of each other a halogen; A may be boron; Y¹ to Y⁴ may be independently of one another fluorine-substituted C6-C12 aryl; and the alkyl of R and the aryl of R¹ to R⁴ may be further substituted by one or more selected from C1-C7alkyl and tric1-C7 alkylsilyl.

In terms of catalytic efficiency, selectivity of ethylene oligomer, and suppression of polyolefin production, more preferably, Chemical Formula 1 may be represented by the following Chemical Formula 4:

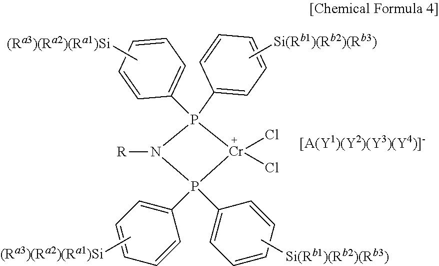

wherein
R is C1-C20 alkyl or C6-C20 aryl;
R^{a1} to R^{a3}, R^{b1} to R^{b3}, R^{c1} to R^{c3}, and R^{d1} to R^{d3} are independently of one another C1-C20 alkyl;
X¹ and X² are independently of each other a halogen, C1-C20 alkyl, C1-C20 alkylcarboxylate, acetylacetonate, or C1-C20 alkoxy;
A is boron or aluminum; and
Y¹ to Y⁴ are independently of one another fluorine-substituted C6-C20 aryl or fluorine-substituted C6-C20 aryloxy.

In an exemplary embodiment, the chromium complex of Chemical Formula 4 may have more improved catalytic activity and ethylene oligomer selectivity, by introducing a trialkylsilyl group as a specific substituent to phenyl (Ph) bonded to phosphorus (P).

Preferably, in Chemical Formula 4 according to an exemplary embodiment, R may be C1-C20 alkyl; X¹ and X² may be independently of each other a halogen, C1-C20 alkyl, C1-C20 alkylcarboxylate, or acetylacetonate; A may be boron; Y¹ to Y⁴ may be independently of one another fluorine-substituted C6-C20 aryl or fluorine-substituted C6-C20 aryloxy, more preferably R may be C1-C10 alkyl; X¹ and X² may be independently of each other a halogen or C1-C10 alkyl; A may be boron; and Y¹ to Y⁴ may be independently of one another fluorine-substituted C6-C12 aryl.

More preferably, in Chemical Formula 4 according to an exemplary embodiment, R may be C1-C5 alkyl; X¹ and X² may be chlorine; A may be boron; Y¹ to Y⁴ may be pentafluorophenyl; and R^{a1} to R^{a3}, R^{b1} to R^{b3}, R^{c1} to R^{c3}, and R^{d1} to R^{d3} may be independently of one another C5-C8 alkyl.

More specifically, the chromium complex according to an exemplary embodiment may be represented by the following Chemical Formula 5:

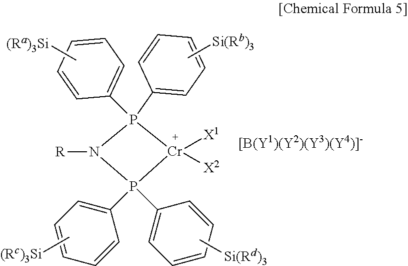

wherein
R is C1-C10 alkyl;
X¹ and X² are independently of each other a halogen, C1-C10 alkyl, or acetylacetonate;
Y¹ to Y⁴ are independently of one another fluorine-substituted C6-C12aryl; and
R^{a} to R^{d} are independently of one another C1-C10 alkyl.

Preferably, in Chemical Formula 5, R^{a} to R^{d} may be all identically C1-C8 alkyl.

In a specific example, in Chemical Formula 5, R may be C1-C5 alkyl; R^{a} to R^{d} may be identical to one another and C5-C8 alkyl, and more specifically R may be branched chain C3-C5 alkyl; and R^{a} to R^{d} may be identical to each other and C5-C8 alkyl.

Still more specifically, in Chemical Formula 5, R may be isopropyl; R^{a} to R^{d} may be all octyl (n-Octyl); and Y¹ to Y⁴ may be all pentafluorophenyl (C6F5). The chromium complex of the structural example as such may further reduce the production amount of a high molecular polyethylene compound as a by-product while further significantly improving activity and selectivity when applied to a catalyst system for an ethylene oligomerization reaction.

In Chemical Formula 2 of the organoaluminum compound according to an exemplary embodiment, R¹¹ to R¹³ may be identical to one another and C4-C8 alkyl, and specifically, may be tri-n-butylaluminum, tri-n-hexylaluminum, or tri-n-octylaluminum.

In Chemical Formula 3 of the organozinc compound according to an exemplary embodiment, R²¹ and R²² may be identical to each other and C1-C4 alkyl, and specifically dimethylzinc or diethylzinc.

In a specific example, the chromium complex, the organoaluminum compound, and the organozinc compound may be a combination of the chromium complex of Chemical Formula 5, tri-n-octylaluminum, and diethylzinc.

In the ethylene oligomerization method according to an exemplary embodiment, the organic solvent may be, though the type is not particularly limited, a hydrocarbon solvent unsubstituted or substituted by a halogen. Specifically, the hydrocarbon solvent may be an aliphatic hydrocarbon solvent having 4 to 20 carbon atoms, an aromatic hydrocarbon solvent having 6 to 20 carbon atoms, a mixture thereof, and the like. More specifically, the hydrocarbon solvent unsubstituted or substituted by a halogen may be toluene, xylene, chlorobenzene, dichlorobenzene, dichloromethane, methylcyclohexene, cyclohexene, and the like, as an example, and preferably, may be dichloromethane, methylcyclohexene, or cyclohexene, and when the exemplified solvent is used, polymerization activity is high and separation of the solvent from 1-hexene and 1-octene which are products after the oligomerization reaction is easier.

In the ethylene oligomerization method according to an exemplary embodiment, in order to maintain process stability by dramatically suppressing production of by-products such as polyolefin while simultaneously maintaining catalytic activity, hydrogen may be further injected before or during injection of ethylene. Herein, a mole ratio between ethylene and hydrogen may be 1:0.001 to 3.0, preferably 1:0.005 to 2.0, more preferably 1:0.01 to 1.0, more preferably 1:0.01 to 0.5, and still more preferably 1:0.01 to 0.2.

In the ethylene oligomerization method according to an exemplary embodiment, the prepared ethylene oligomer is 1-hexene, 1-octene, or a mixture thereof, and the content of polyethylene may be less than 0.1 wt %, specifically 0.07 wt % or less, 0.05 wt % or less, 0.03 wt % or less, or 0.2 wt % or less.

The ethylene oligomerization method according to an exemplary embodiment has high activity and selectivity and also suppresses production of polyethylene to maintain process stability, through a specific combination of the chromium complex, the organoaluminum compound, and the organozinc compounds and optimization of the conditions of reaction temperature and reaction pressure, and the injection amount of the organozinc compound.

Hereinafter, the present invention will be described in more detail through the following Examples. However, these examples are for illustrative purposes only, and the present invention is not limited to the examples.

### Preparation Example 1

A chromium complex represented by the following Chemical Formula A was prepared according to the following method.

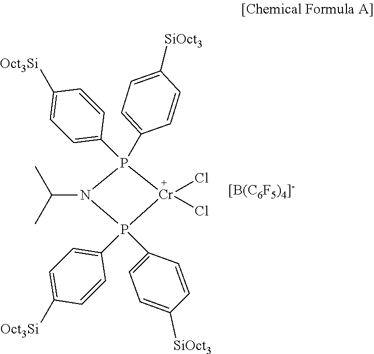

### Synthesis of C1-Si(n-Octyl)₃

A solution in which acetyl chloride (1.54 g, 19.6 mmol) was dissolved in CH₂C12(7 mL) was added dropwise to a CH₂C12 (20 mL) solution including trioctylsilane (4.83 g, 13.1 mmol) and FeCl₃ (0.0549 g, 0.262 mmol). As FeCl₃ was dissolved, it was confirmed that the color of the solution turned yellow with heating, and then the solution was stirred at room temperature for 24 hours. The solvent, acetaldehyde as a by-product, and acetyl chloride as an unreacted material were removed using a vacuum line. The residue was dissolved in hexane (10 mL) and an undissolved brown solid (FeCl₃) was removed by filtration (Celite-aided filtration). The solvent was removed using a vacuum line to obtain a light yellow oil compound (4.98 g, 98%).

### Synthesis of BrC₆H₄-p-Si(n-Octyl)₃

1, 4-Dibromobenzene (3.31 g, 14.0 mmol) was dissolved in THE (35 mL), n-butyllithium (5 mL, 2.5 M hexane solution, 12.5 mmol) was injected at −78° C., and then stirring was performed for 2 hours while the temperature was maintained at −78° C. A solution of C1-Si(n-Octyl)₃ (4.79 g, 11.9 mmol) dissolved in THE (6 mL) was injected, the temperature was raised to room temperature, and the reaction was performed for 3 hours. The solvent was removed using a vacuum line, the product to be desired was dissolved in hexane (18 mL), and an insoluble white solid (LiBr) was removed by filtration (celite-aided filtration). The solvent was removed from the filtered liquid using a vacuum line, and the filtrate was dissolved again in hexane (18 mL) and passed through silica gel (short pad of silica gel, 6.22 g). The solvent was removed using a vacuum line, and the residue was distilled in vacuum at 80° C. to remove an unreacted material (1, 4-dibromobenzene) to obtain oil as a target compound (5.71 g, 92%).

### Synthesis of CIP [C6H₄-p-Si(n-Octyl)_{3] 2}

BrC₆H₄-p-Si(n-Octyl)₃ (5.71 g, 10.9 mmol) was dissolved in THF (39 mL), n-butyllithium (4.36 mL, 2.5 M hexane solution, 10.9 mmol) was injected at −78° C., and then stirring was performed for 1 hour while the temperature was maintained at −78° C. Dichloro (diethylamino)phosphine (0.949 g, 5.45 mmol) dissolved in THE (9 mL) was injected for 15 minutes, the temperature was raised to 5° C., and the reaction was performed for 2 hours while maintaining the temperature. Methylcyclohexane (19 mL) was injected, the solvent was removed at room temperature using a vacuum line, methylcyclohexane (31 mL) was added, and an insoluble white solid (LiBr and LiCl) was removed by filtration (celite-aided filtration). The solvent was removed, PCl₃ (4.12 g, 30.0 mmol) was added, and the reaction was performed at 70° C. for 2 hours. Vacuum distillation was performed at 80° C. to remove PCl₃ as an unreacted material and dichloro (diethylamino)phosphine as a by-product, thereby obtaining a compound of yellow oil. The oil compound was dissolved in hexane (23 mL), and an undissolved by-product was removed by filtration (celite-aided filtration). The solvent was removed through a vacuum line to obtain a target compound as yellow oil (5.18 g, 99%).

### Synthesis of i-Propyl N[P(C6H₄-p-Si(n-Octyl)₃)_{2] 2}

A solution in which i-PrNH₂(0.135 g, 2.28 mmol) was dissolved in CH₂C12(11 mL) was added dropwise to a CH₂C12(19 mL) solution including CIP [C6H₄-p-Si(n-Octyl)_{3] 2}(4.79 g, 5.02 mmol) and Et₃N(2.31 g, 22.8 mmol). The reactant was stirred at room temperature for 12 hours, and a volatile component was removed with a vacuum line. Hexane (40 mL) was added to the residue, and (Et₃NH)⁺C1⁻ as an insoluble by-product was removed by filtration (celite-aided filtration). The filtered liquid was passed through silica gel (short pad of silica gel) which was pretreated with hexane/Et₃N(v/v, 50:1), and then the solvent was removed using a vacuum line to obtain a resulting product in the form of a colorless oil (4.26 g, 98%).

### Synthesis of [(i-propyl N[P(C₆H₄-p-Si(n-Octyl)₃)₂]₂)-CrCl₂]⁺[B(C₆F₅)₄]⁻

A solution in which i-propyl N[P(C₆H₄-p-Si(n-Octyl)₃)_{2] 2} (1.41 g, 0.742 mmol) was dissolved in CH₂Cl₂(13 mL) was added dropwise to a solution prepared by dissolving [CrCl₂(NCCH₃)₄]⁺[B(C₆F₅)₄]⁻(0.715 g, 0.742 mmol) in CH₂Cl₂(4.5 mL). The reactant was stirred at room temperature for 2.5 hours, and the solvent was removed with a vacuum line to obtain a viscous green oil.

The obtained oil was dissolved in methylcyclohexane (5 mL) and the solvent was removed with a vacuum line. The process was repeated until CH₃CN and CH₃Cl₂ were completely removed to obtain a resulting product in the form of a viscous green oil (2 g, 100%).

### [Example 1] Ethylene Oligomerization Reaction

The chromium complex of Chemical Formula A prepared in Preparation Example 1 was dissolved in methylcyclohexane (23.4 mL) to prepare a 10 wt % solution, which was used in ethylene oligomerization.

25 ml/min of methylcyclohexane, 0.2 ml/min of tri-n-octyloaluminum (TnOA), 0.2 ml/min of diethylzinc (DEZ), and the chromium complex of Chemical Formula A prepared in Preparation Example 1 were continuously injected into a 2 L autoclave reactor heated to 40° C. The chromium complex was injected at a concentration of 1.1 μM. The reaction temperature was maintained at 40° C. while ethylene and hydrogen were continuously supplied so that the reactor internal pressure was maintained at 30 bar, and a continuous experiment was performed for 90 hours. At this time, a mole ratio between ethylene and hydrogen (ethylene:hydrogen) was maintained at 100:1.

For product analysis, the contents of produced oligomers {1-octene (1-C8), 1-hexene (1-C6), methylcyclopentane-+methylenecyclopentane (cy-C6), and higher oligomers above C10 (>C10)} were measured by gas chromatography (GC) analysis, and the weight ratio of the product was calculated. The produced solid polyethylene was separated by filtration at room temperature, the weight was measured, and the wt % of polyethylene was calculated by the formula of [weight (g) of produced PE/total weight (g) of product].

### [Example 2] Ethylene Oligomerization Reaction

The ethylene oligomerization reaction was performed in the same manner as in Example 1, except that the reaction temperature was 50° C.

### [Example 3] Ethylene Oligomerization Reaction

The ethylene oligomerization reaction was performed in the same manner as in Example 1, except that the reaction temperature was 60° C.

### [Example 4] Ethylene Oligomerization Reaction

The ethylene oligomerization reaction was performed in the same manner as in Example 1, except that the reaction temperature was 60° C., and the flow rate of diethylzinc (DEZ) was 0.15 ml/min.

### [Example 5] Ethylene Oligomerization Reaction

The ethylene oligomerization reaction was performed in the same manner as in Example 1, except that the reaction temperature was 60° C., and the flow rate of methylcyclohexane was 20 ml/min.

### [Example 6] Ethylene Oligomerization Reaction

The ethylene oligomerization reaction was performed in the same manner as in Example 1, except that the reaction temperature was 60° C., the flow rate of methylcyclohexane was 20 ml/min, and the flow rate of diethylzinc (DEZ) was 0.3 ml/min.

### [Comparative Example 1] Ethylene Oligomerization Reaction

The ethylene oligomerization reaction was performed in the same manner as in Example 1, except that diethylzinc (DEZ) was not injected.

Activities of the olefin polymerization reactions and the compositions of the prepared polymers of Examples 1 to 6 and Comparative Example 1 are shown in the following Table 1.

TABLE 1

| | Al/Cr | Zr/Cr | MCH | Temperature | LAO | Product composition (wt %) | | | | |
|---|---|---|---|---|---|---|---|---|---|---|
| | ratio | ratio | (ml/min) | (° C.) | (%) | 1-C6 | Cy-C6 | 1-C8 | C10+ | PE |
| Example 1 | 200 | 700 | 25 | 40 | 17 | 11.50 | 5.30 | 71.30 | 11.89 | 0.01 |
| Example 2 | 200 | | | 50 | 28 | 16.90 | 4.90 | 66.40 | 11.79 | 0.01 |
| Example 3 | 200 | | | 60 | 29 | 24.00 | 4.40 | 58.90 | 12.68 | 0.02 |
| Example 4 | 200 | 525 | | 60 | 29 | 24.00 | 4.40 | 60.50 | 11.05 | 0.05 |
| Example 5 | 200 | 700 | 20 | 60 | 35 | 24.50 | 4.40 | 57.70 | 13.33 | 0.07 |
| Example 6 | 200 | 1050 | | 60 | 35 | 24.30 | 4.40 | 57.90 | 13.37 | 0.03 |
| Comparative | 200 | 0 | 25 | 40 | 15 | 11.80 | 5.30 | 70.90 | 11.38 | 0.62 |
| Example 1 | | | | | | | | | | |

As shown in Table 1, Examples 1 to 6 to which DEZ was injected had better activity, and also had a polyethylene production amount decreased by 95% or more as compared with Comparative Example 1 to which DEZ was not injected.

As the reaction temperature rose, the activity was increased, and a LAO concentration was increased to 35% at 60° C. (Examples 1 to 5). As a LAO concentration was increased, the polymer production amount was increased to 0.07% (Example 5), but when a DEZ injection amount was increased, the polymer production amount was decreased by 50% or more (Example 6).

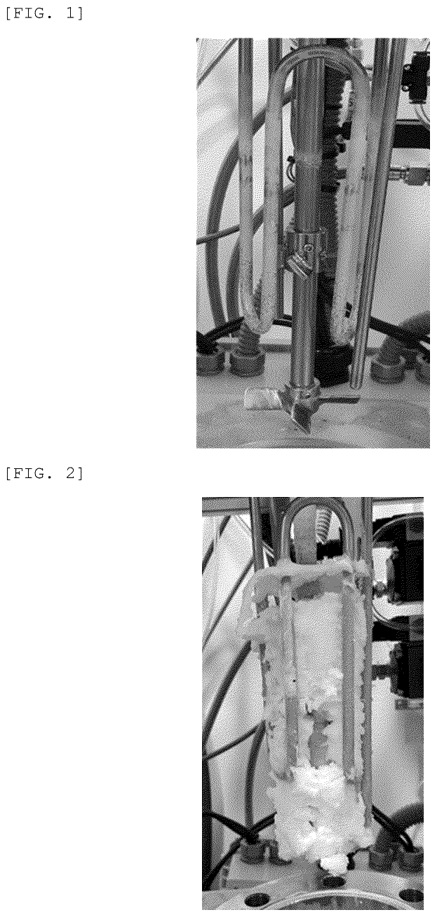

In addition, after the reaction was finished, a difference in polyethylene accumulated in the reactor was clearly confirmed from FIG. 1 (Example 1) and FIG. 2 (Comparative Example 1).

The ethylene oligomerization method according to an exemplary embodiment of the present invention may produce an ethylene oligomer with high catalytic activity and excellent selectivity and also may significantly decrease a polyethylene production amount, by optimizing the injection amount of an organozinc compound when reacting a chromium complex, an organoaluminum compound, and an organozinc compound with ethylene to perform an oligomerization reaction at a constant temperature, and thus, pipe blockage and fouling do not occur to allow long-term continuous production, which is very advantageous for commercialization.

The spirit of the present disclosure has been just exemplified, and it will be appreciated by those skilled in the art that various modifications and alterations can be made without departing from the spirit of essential characteristics of the present invention. In addition, the present exemplary embodiments are not for limiting but for describing the spirit of the present disclosure, and the scope of the spirit of the present disclosure is not limited by the exemplary embodiment. The scope of the present disclosure should be interpreted by the following claims and it should be interpreted that all spirits equivalent to the following claims fall within the scope of the present disclosure.

1. An ethylene oligomerization method comprising reacting a chromium complex represented by the following Chemical Formula 1, an organoaluminum compound represented by the following Chemical Formula 2, and an organozinc compound represented by the following Chemical Formula 3 with ethylene in the presence of an organic solvent to prepare an ethylene oligomer,
wherein a mole ratio (Cr:Zn) between the chromium complex and the organozinc compound is 1:100 to 1500, and the reaction is performed at a temperature of 0 to 80° C. under a pressure of 1 to 100 bar: 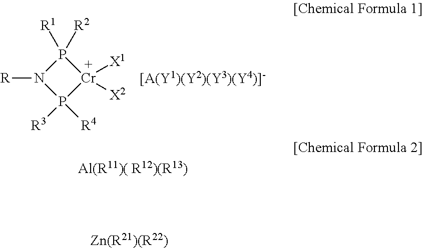
Wherein
R is C1-C60 alkyl, C6-C60 aryl, or C2-C60 heteroaryl;
R¹ to R⁴ are independently of one another C1-C60 alkyl, C6-C60 aryl, or C2-C60 heteroaryl;
X¹ and X² are independently of each other a halogen, C1-C30 alkyl, C1-C30 alkylcarboxylate, acetylacetonate, or C1-C30 hydrocarbyl including one or more selected from ether and amino;
A is boron or aluminum;
Y¹ to Y⁴ are independently of one another fluorine-substituted C6-C60 aryl, fluorine-substituted C2-C60 heteroaryl, fluorine-substituted C6-C60 aryloxy, or fluorine-substituted C6-C60 alkoxy;
R¹¹ to R¹³ are independently of one another C4-C8 alkyl;
R²¹ to R²² are independently of each other C1-C8 alkyl;
the alkyl, aryl, or heteroaryl of R and R¹ to R⁴ may be further substituted by one or more selected from C1-C30 alkyl, C6-C30 aryl, C1-C30 alkoxy, monoC1-C30 alkylamino, diC1-C30 alkylamino, monoC6-C30 arylamino, diC6-C30 arylamino, monoC1-C30 alkylsilyl, diC1-C30 alkylsilyl, triC1-C30 alkylsilyl, monoC6-C30 arylsilyl, diC6-C30 arylsilyl, and triC6-C30 arylsilyl; and
the heteroaryl includes at least one heteroatom selected from the group consisting of O, N, S, Si, and P.

2. The ethylene oligomerization method of claim 1, wherein the chromium complex is represented by the following Chemical Formula 4: 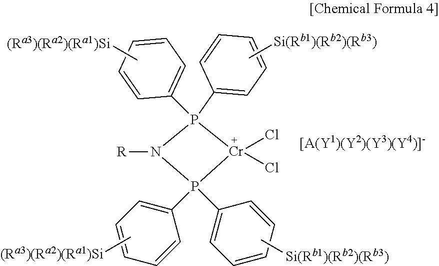
wherein
R is C1-C20 alkyl or C6-C20 aryl;
R^{a1} to R^{a3}, R^{b1} to R^{b3}, R^{c1} to R^{c3}, and R^{d1} to R^{d3} are independently of one another C1-C20 alkyl;
X¹ and X² are independently of each other a halogen, C1-C20 alkyl, C1-C20 alkylcarboxylate, acetylacetonate, or C1-C20 alkoxy;
A is boron or aluminum; and
Y¹ to Y⁴ are independently of one another fluorine-substituted C6-C20 aryl or fluorine-substituted C6-C20 aryloxy.

3. The ethylene oligomerization method of claim 1,
wherein in Chemical Formula 2, R¹¹ to R¹³ are identical to one another and C4-C8 alkyl; and
in Chemical Formula 3, R²¹ and R²² are identical to each other and C1-C4 alkyl.

4. The ethylene oligomerization method of claim 3,
wherein the organoaluminum compound of Chemical Formula 2 is tri-n-butylaluminum, tri-n-hexylaluminum, or tri-n-octylaluminum; and
the organozinc compound of Chemical Formula 3 is dimethylzinc or diethylzinc.

5. The ethylene oligomerization method of claim 1, wherein the reaction is performed at a temperature of 40 to 60° C. under a pressure of 20 to 40 bar.

6. The ethylene oligomerization method of claim 1, wherein a mole ratio (Cr:Al) between the chromium complex and the organoaluminum compound is 1:100 to 500.

7. The ethylene oligomerization method of claim 1, wherein a mole ratio (Cr:Zn) between the chromium complex and the organozinc compound is 1:500 to 1300.

8. The ethylene oligomerization method of claim 1, wherein the ethylene oligomerization method is performed in a continuous stirred tank reactor (CSTR).

9. The ethylene oligomerization method of claim 1, wherein the ethylene oligomerization method further includes hydrogen.

10. The ethylene oligomerization method of claim 9, wherein a mole ratio between ethylene and hydrogen is 1:0.001 to 3.0.

11. The ethylene oligomerization method of claim 1, wherein the ethylene oligomer is 1-hexene, 1-octene, or a mixture thereof, and a polyethylene content is less than 0.1 wt %.
